(19) United States
(12) Patent Application Publication
(10) Pub. No.: US 2026/0370201 A1
(43) Pub. Date: Nov. 19, 2026

(54) INTERLEAVED AMPLIFIER FOR SENSING SYSTEM

(71) Applicant: Cirrus Logic International Semiconductor Ltd., Edinburgh (GB)

(72) Inventors: Lei ZHU, Austin, TX (US); Johann G. GABORIAU, Austin, TX (US); Xin ZHAO, Austin, TX (US); Xiaofan FEI, Austin, TX (US); John L. MELANSON, Austin, TX (US); Wai-Shun SHUM, Austin, TX (US); Thomas JANES, Austin, TX (US); Alan M. MORTON, Austin, TX (US)

(73) Assignee: Cirrus Logic International Semiconductor Ltd., Edinburgh (GB)

(21) Appl. No.: 19/655,040

(22) Filed: Apr. 22, 2026

Related U.S. Application Data
(60) Provisional application No. 63/806,846, filed on May 16, 2025.

(51) Int. Cl. H03G 3/30; A61B 5/30; H03F 3/45

(52) U.S. Cl. CPC H03G 3/30; A61B 5/30; H03F 3/45475; H03G 2201/103

(57) ABSTRACT

An interleaved amplifier for a sensing system may include an analog multiplexer (MUX) configured to receive a sensed signal from a sensor, a first amplifier stage coupled to the analog MUX, the first amplifier stage comprising N amplifiers, wherein N is an integer of two or greater, and wherein each of the N amplifiers is operable in an amplification mode and a sample and hold mode, a sample circuit block coupled to the first amplifier stage and configured to sample an output of a selected amplifier of the N amplifiers, and a second amplifier stage comprising a hold amplifier block coupled to the sample circuit block, wherein the analog MUX is configured to select operation of only a single amplifier of the N amplifiers at a time such that the first amplifier stage operates as an interleaved amplifier providing increased bandwidth and reduced power consumption relative to a non-interleaved amplifier configuration.

### CROSS-REFERENCES AND RELATED APPLICATION

The present disclosure claims benefit of U.S. Provisional Patent Application Ser. No. 63/806,846, filed May 16, 2025, which is incorporated by reference herein in its entirety.

### FIELD OF DISCLOSURE

The present disclosure relates in general to circuits for electronic devices, including without limitation smart devices configured for monitoring activity such as health monitoring, and more specifically, to systems and methods for providing an interleaved amplifier for a sensing system in which an amplifier stage of the sensing system provides increased bandwidth, improved gain accuracy, reduced gain drift, and reduced power consumption relative to existing approaches.

### BACKGROUND

Personal electronic devices, such as smart devices, are ubiquitous in modern society and are utilized by users for a variety of purposes. Such smart devices may be configured for monitoring activities, including health monitoring, to provide useful information to the user.

A sensing system may be implemented to perform such monitoring activity in a smart device. Such a sensing system typically includes an analog front end (AFE) for sensing analog signals. A sensing system of this type may typically require a first amplifier stage for amplifying the sensed analog signal and a second amplifier stage for sampling and holding the analog signal.

Conventional sensing systems may present challenges with respect to achieving very tight gain accuracy and very low gain drift with wide input current range in the first amplifier stage. Furthermore, the first amplifier stage may require higher power consumption, which can be undesirable in portable electronic devices where battery life is a concern. In addition, conventional approaches using continuous time transimpedance amplifiers (TIAs), particularly for current-mode sensors such as photodiodes, may face difficulties achieving tight gain accuracy and low gain drift specifications. Such TIAs may use capacitor-based integrators that require periodic reset, but the reset time can adversely affect system performance. In current-mode sensing systems such as photodiodes, first-stage transimpedance amplifiers must meet stringent gain accuracy and gain drift specifications across process and temperature. Conventional continuous-time integrator-based TIAs require periodic reset, and the reset interval introduces gain error, bandwidth loss, and signal distortion. Accordingly, there is a need for a sensing system in which the first amplifier stage achieves improved gain accuracy, reduced gain drift, and wider input current range while consuming less power, as compared to existing approaches.

### SUMMARY

In accordance with the teachings of the present disclosure, one or more disadvantages and problems associated with existing approaches to sensing systems may be reduced or eliminated.

In accordance with embodiments of the present disclosure, an interleaved amplifier for a sensing system may include an analog multiplexer (MUX) configured to receive a sensed signal from a sensor, a first amplifier stage coupled to the analog MUX, the first amplifier stage comprising N amplifiers, wherein N is an integer of two or greater, and wherein each of the N amplifiers is operable in an amplification mode and a sample and hold mode, a sample circuit block coupled to the first amplifier stage and configured to sample an output of a selected amplifier of the N amplifiers, and a second amplifier stage comprising a hold amplifier block coupled to the sample circuit block, wherein the analog MUX is configured to select operation of only a single amplifier of the N amplifiers at a time such that the first amplifier stage operates as an interleaved amplifier providing increased bandwidth and reduced power consumption relative to a non-interleaved amplifier configuration.

In accordance with these and other embodiments of the present disclosure, a method for operating an interleaved amplifier in a sensing system may include receiving, by an analog multiplexer (MUX), a sensed signal from a sensor, selectively routing, by the analog MUX, the sensed signal to a selected amplifier of N amplifiers in a first amplifier stage, wherein N is an integer of two or greater, and wherein each of the N amplifiers is operable in an amplification mode and a sample and hold mode, sampling, by a sample circuit block coupled to the first amplifier stage, an output of the selected amplifier, holding, by a second amplifier stage comprising a hold amplifier block coupled to the sample circuit block, the sampled output, and selecting, by the analog MUX, operation of only a single amplifier of the N amplifiers at a time such that the first amplifier stage operates as an interleaved amplifier providing increased bandwidth and reduced power consumption relative to a non-interleaved amplifier configuration.

Technical advantages of the present disclosure may be readily apparent to one skilled in the art from the figures, description and claims included herein. The objects and advantages of the embodiments will be realized and achieved at least by the elements, features, and combinations particularly pointed out in the claims.

It is to be understood that both the foregoing general description and the following detailed description are examples and explanatory and are not restrictive of the claims set forth in this disclosure.

### BRIEF DESCRIPTION OF THE DRAWINGS

A more complete understanding of the present embodiments and advantages thereof may be acquired by referring to the following description taken in conjunction with the accompanying drawings, in which like reference numbers indicate like features, and wherein:

FIG. 1 illustrates a block diagram of selected components of an example sensing system having an interleaved amplifier, in accordance with embodiments of the present disclosure;

FIG. 2A illustrates a circuit diagram of an exemplary embodiment of gain selection for a first feedback path of the interleaved amplifier of FIG. 1, in accordance with embodiments of the present disclosure;

FIG. 2B illustrates a circuit diagram of another exemplary embodiment of gain selection for a first feedback path of the interleaved amplifier of FIG. 1, in accordance with embodiments of the present disclosure; and

FIG. 3 illustrates a circuit diagram of an exemplary anti-aliasing filtering for a second feedback path for a second amplifier stage of the interleaved amplifier of FIG. 1, in accordance with embodiments of the present disclosure.

### DETAILED DESCRIPTION

Embodiments of the present disclosure provide a system and method of an interleaved amplifier that may be utilized for a sensing system.

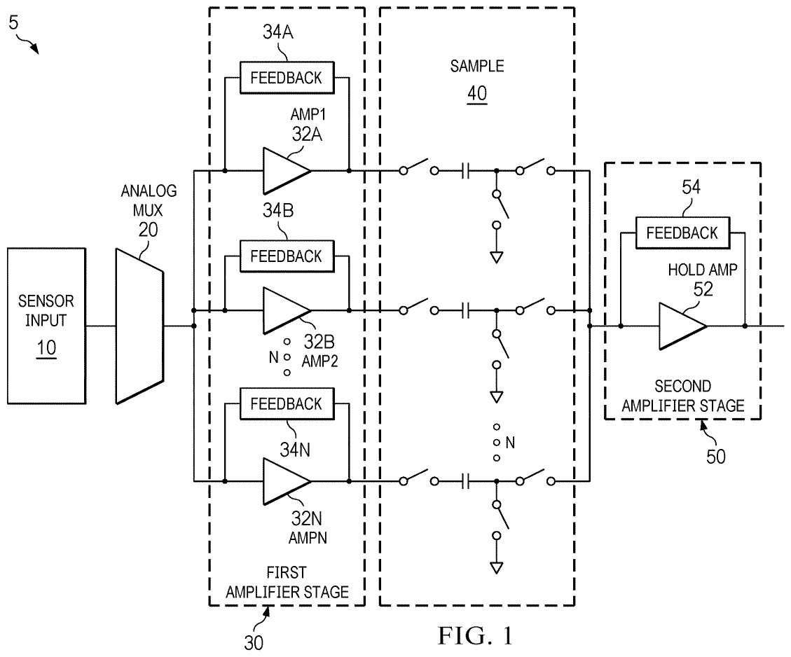

FIG. 1 illustrates a block diagram of selected components of an example sensing system 5 having an interleaved amplifier, in accordance with embodiments of the present disclosure. As shown in FIG. 1, sensing system 5 may comprise a sensor input 10, an analog multiplexer (MUX) 20, a first amplifier stage 30, a sample circuit block 40, and a second amplifier stage 50.

Sensor input 10 may comprise any system, device, or apparatus configured to detect and generate an electronic sensed signal representing a physical parameter or physiological activity. In certain embodiments, sensor input 10 may comprise a biopotential sensor for health monitoring applications, such as an electrocardiogram (ECG) sensor, an electromyography (EMG) sensor, an electroencephalography (EEG) sensor, or other suitable sensor for detecting electrical signals generated by the body. In some primary embodiments, the sensor may comprise a photodiode and the first amplifier stage operates as a current-input transimpedance amplifier, wherein gain accuracy and drift directly impact optical sensing accuracy. The sensed signal generated by sensor input 10 may be provided as an input to analog MUX 20 for subsequent processing by sensing system 5.

Analog MUX 20 may comprise any system, device, or apparatus configured to receive the sensed signal from sensor input 10 and selectively route the signal to one of N number of amplifiers in first amplifier stage 30. For example, for a voltage input signal, analog MUX 20 may comprise N number of switches to selectively direct the sensed signal to the appropriate amplifier of first amplifier stage 30. The On/Off control signal of each switch in analog MUX 20 may be overlapped as the input signal will be eventually added together at the second amplifier stage 50.

First amplifier stage 30 may comprise an array of a number N of amplifiers 32A, 32B, . . . , 32N, wherein N is an integer of two or greater. In certain embodiments, N may equal two, which may provide sufficient performance benefits while minimizing circuit complexity. In other embodiments, N may equal three or greater for additional performance improvement. Amplifiers 32A, 32B, . . . , 32N may be individually referred to herein as an “amplifier 32” and collectively referred to herein as “amplifiers 32.” Each amplifier 32 may be implemented as an operational amplifier, transimpedance amplifier, or other suitable amplification circuit configured to receive a single-ended or differential input signal and generate an amplified output signal. Amplification by an amplifier 32 may be achieved through integrating input current or applying a voltage gain, and such integration operation may be implemented with a first feedback path 34 (e.g., first feedback paths 34A, 34B, . . . , 34N) for such amplifier 32. Such amplifier configuration may allow first amplifier stage 30 to accommodate various types of sensor inputs while providing the amplification necessary for subsequent processing by sample circuit block 40 and second amplifier stage 50.

The interleaved amplifier architecture of first amplifier stage 30 may provide advantages over conventional continuous-time TIA implementations. Conventional TIAs using capacitor-based integrators may require periodic reset operations, and the reset time can negatively impact system performance. By interleaving multiple amplifiers 32, the system may allow one amplifier to undergo reset while another amplifier continues to process the input signal, thereby interleaving masks reset-induced gain error and sampling discontinuity by time-multiplexing multiple integrators such that at least one integrator continuously processes the input signal while another undergoes reset or leakage control. Additionally, the interleaved architecture may enable improved gain accuracy and reduced gain drift compared to conventional approaches, which may be particularly advantageous for sensing applications requiring tight gain specifications.

A fully resetting integrator may introduce dead time and signal discontinuities, while a leaky integrator may reduce direct current (DC) gain and introduce controlled current bleed. The present disclosure recognizes that interleaving multiple integrators allows one integrator to reset or leak while another continues amplification, thereby decoupling reset behavior from signal acquisition.

Each amplifier 32 may have two operation modes: an amplification mode and a sample and hold/irrelevant mode. In the amplification mode, an input voltage or current, which is an output from analog MUX 20, may be amplified, and the amplified signal may be provided at the output of such amplifier 32. In a sample mode, the output of an amplifier 32 may first be sampled by sample circuit block 40 and then such amplifier 32 may transition to any mode because its output does not impact system performance.

Sample circuit block 40 may comprise any system, device, or apparatus configured to capture and store the amplified output signal from a currently-active amplifier 32 of first amplifier stage 30. In operation, sample circuit block 40 may sample the output of a single selected amplifier 32 during the sample mode and transfer the sampled signal to second amplifier stage 50 for subsequent hold and processing operations.

Second amplifier stage 50 may be configured as a hold amplifier block that receives and holds the sampled signals from sample circuit block 40. In operation, second amplifier stage 50 may add together the sampled outputs from the N number of amplifiers of first amplifier stage 30, thereby combining the interleaved amplifier outputs into a unified signal. Second amplifier stage 50 may comprise an amplifier 52, which may be implemented as an operational amplifier or other suitable amplification circuit configured to receive a single-ended or differential input signal and generate an amplified output signal. Amplification by amplifier 52 may be achieved through integrating input current or applying a voltage gain, and such integration operation may be implemented with a second feedback path 54. Further, in the hold/irrelevant mode of second amplifier stage 50, second amplifier stage 50 may be able to reset or switch operations of a selected amplifier 32 to a low power mode.

In operation, an interface to the input of each amplifier 32 may be a single-ended or differential input signal. The input signal may be either a voltage signal or a current signal. For a voltage input signal, analog MUX 20 may comprise two or more N number of resistors in which each resistor is in series with a respective switch that is connected to the input of analog MUX 20 and only one switch is on at a time to activate only a single amplifier 32 at a time. In other words, first amplifier stage 30 may have an interleaved amplifier operation that makes it an interleaved amplifier because analog MUX 20 may only select operation of a single amplifier 32 at a time. Analog MUX 20 may further convert the voltage input signal to a current input signal.

For a current input signal, analog MUX 20 may comprise two or more N number of switches that are connected to the input of analog MUX 20 and only one switch may be on at a time to activate only a single amplifier 32 at a time. Similar to a voltage input signal, a current input signal implementation may also comprise first amplifier stage 30 having an interleaved amplifier operation that makes it an interleaved amplifier because analog MUX 20 only selects operation of a single amplifier 32 at a time.

Each amplifier 32 may have a fixed gain or a programmable gain in its respective feedback path 34. In the case of a programmable gain, feedback path 34 may comprise multiple gain switching elements, with each gain switching element having a switch in series with a resistor or capacitor. The gain switching element may be rotated by a dynamic element matching (DEM) block if feedback path 34 is a switch in series with a capacitor implementation. For every clock cycle of such capacitor implementation, the DEM block may generate a new pattern and randomly pick one or multiple capacitors for charging, and all the gain switching elements may be used in any single gain setting, which may maintain a constant gain ratio among programmable gain settings.

In certain embodiments, the feedback path may implement an effective resistance using a switched-capacitor network updated once per cycle, thereby forcing a deterministic DC gain based on capacitor ratios independent of absolute resistance values.

In certain embodiments, each amplifier 32 may be implemented as a leaky integrator. A leaky integrator may provide a fixed, known gain at DC based on a capacitor ratio, which may reduce the DC response while maintaining signal bandwidth at frequencies of interest. This approach may be advantageous because sensing applications may not require signal bandwidth at DC. In other embodiments, each amplifier 32 may be periodically reset rather than configured as a leaky integrator, and the interleaved structure may mitigate the performance impact of such reset operations by allowing continuous signal processing through the other amplifiers of first amplifier stage 30.

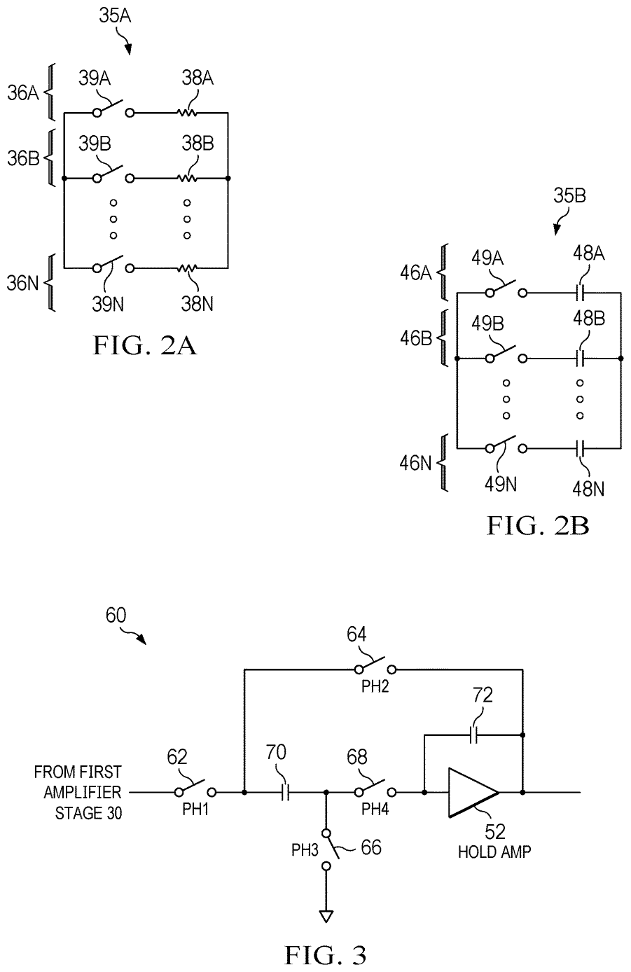

A filtering function may be implemented in second amplifier stage 50, such as the anti-aliasing filter depicted in FIG. 3. Examples of programmable gain selection implementations for first amplifier stage 30 are depicted in FIGS. 2A and 2B. These implementations are further described below.

The interleaved amplifier architecture may also provide temperature compensation advantages. By using dynamic element matching (DEM) with capacitor-based gain switching elements as described herein, the system may achieve improved gain matching between different operating modes, which may reduce temperature-dependent variations in gain (temperature coefficient). This temperature compensation benefit may be particularly valuable in portable devices that operate across a range of environmental conditions.

By distributing amplification across interleaved paths, the required transconductance of each amplifier may be reduced, enabling lower bias current and improved power efficiency.

FIG. 2A illustrates a circuit diagram of an exemplary embodiment of programmable gain selection 35A which may be used in a feedback path 34 of first amplifier stage 30, in accordance with embodiments of the present disclosure. As shown in FIG. 2A, programmable gain selection 35A may comprise a plurality of series paths 36 (e.g., series paths 36A, 36B, . . . , 36N), each series path 36 comprising a respective feedback resistor 38 (e.g., feedback resistors 38A, 38B, . . . , 38N) and a respective gain switching element 39 (e.g., gain switching elements 39A, 39B, . . . , 39N). Each series path 36 in programmable gain selection 35A may be in parallel to the other series paths 36, as shown in FIG. 2A.

FIG. 2B illustrates a circuit diagram of an exemplary embodiment of programmable gain selection 35B which may be used in a feedback path 34 of first amplifier stage 30, in accordance with embodiments of the present disclosure. As shown in FIG. 2B, programmable gain selection 35B may comprise a plurality of series paths 46 (e.g., series paths 46A, 46B, . . . , 46N), each series path 46 comprising a respective feedback capacitor 48 (e.g., feedback capacitors 48A, 48B, . . . , 48N) and a respective gain switching element 49 (e.g., gain switching elements 49A, 49B, . . . , 49N). Each series path 46 in programmable gain selection 35B may be in parallel to the other series paths 46, as shown in FIG. 2B.

FIG. 3 illustrates a circuit diagram of an example anti-aliasing filter (AAF) 60 for second amplifier stage 50, in accordance with embodiments of the present disclosure. As shown in FIG. 3, the AAF 60 may comprise a switched capacitor low pass filter having switching elements 62, 64, 66, and 68, a sampling capacitor 70, a holding capacitor 72, and a holding amplifier 52 coupled together in the manner shown. Together, elements of AAF 60 shown in FIG. 3 may implement sample circuit block 40 and second amplifier stage 50.

As used herein, when two or more elements are referred to as “coupled” to one another, such term indicates that such two or more elements are in electronic communication or mechanical communication, as applicable, whether connected indirectly or directly, with or without intervening elements.

This disclosure encompasses all changes, substitutions, variations, alterations, and modifications to the example embodiments herein that a person having ordinary skill in the art would comprehend. Similarly, where appropriate, the appended claims encompass all changes, substitutions, variations, alterations, and modifications to the example embodiments herein that a person having ordinary skill in the art would comprehend. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative. Accordingly, modifications, additions, or omissions may be made to the systems, apparatuses, and methods described herein without departing from the scope of the disclosure. For example, the components of the systems and apparatuses may be integrated or separated. Moreover, the operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set.

Although exemplary embodiments are illustrated in the figures and described below, the principles of the present disclosure may be implemented using any number of techniques, whether currently known or not. The present disclosure should in no way be limited to the exemplary implementations and techniques illustrated in the drawings and described above.

Unless otherwise specifically noted, articles depicted in the drawings are not necessarily drawn to scale.

All examples and conditional language recited herein are intended for pedagogical objects to aid the reader in understanding the disclosure and the concepts contributed by the inventor to furthering the art, and are construed as being without limitation to such specifically recited examples and conditions. Although embodiments of the present disclosure have been described in detail, it should be understood that various changes, substitutions, and alterations could be made hereto without departing from the spirit and scope of the disclosure.

Although specific advantages have been enumerated above, various embodiments may include some, none, or all of the enumerated advantages. Additionally, other technical advantages may become readily apparent to one of ordinary skill in the art after review of the foregoing figures and description.

Further, reciting in the appended claims that a structure is “configured to” or “operable to” perform one or more tasks is expressly intended not to invoke 35 U.S.C. § 112(f) for that claim element. Accordingly, none of the claims in this application as filed are intended to be interpreted as having means-plus-function elements. Should Applicant wish to invoke § 112(f) during prosecution, Applicant will recite claim elements using the “means for [performing a function]” construct.

What is claimed is:

1. An interleaved amplifier for a sensing system, comprising:
an analog multiplexer (MUX) configured to receive a sensed signal from a sensor;
a first amplifier stage coupled to the analog MUX, the first amplifier stage comprising N amplifiers, wherein N is an integer of two or greater, and wherein each of the N amplifiers is operable in an amplification mode and a sample and hold mode;
a sample circuit block coupled to the first amplifier stage and configured to sample an output of a selected amplifier of the N amplifiers; and
a second amplifier stage comprising a hold amplifier block coupled to the sample circuit block;
wherein the analog MUX is configured to select operation of only a single amplifier of the N amplifiers at a time such that the first amplifier stage operates as an interleaved amplifier providing increased bandwidth and reduced power consumption relative to a non-interleaved amplifier configuration.

2. The interleaved amplifier for a sensing system of claim 1, wherein the first amplifier stage is configured to reduce gain at direct current (DC) while maintaining gain within a target signal bandwidth.

3. The interleaved amplifier for a sensing system of claim 1, wherein in the amplification mode, an input current or voltage from the analog MUX is amplified by the selected single amplifier, and wherein amplification by the selected single amplifier is achieved through integrating the input current or applying a voltage gain, the integration operation for the input current being implemented with a first feedback path of the selected single amplifier.

4. The interleaved amplifier for a sensing system of claim 3, wherein in the sample and hold mode, an output of the selected single amplifier is sampled by the sample circuit block, and wherein the selected single amplifier transitions to a mode in which its output does not impact system performance after sampling.

5. The interleaved amplifier for a sensing system of claim 4, wherein in a hold mode of the sample and hold mode, the second amplifier stage is configured to reset or switch operations of the selected single amplifier to a low power mode.

6. The interleaved amplifier for a sensing system of claim 3, wherein each amplifier of the N amplifiers has a fixed gain or a programmable gain implemented in the first feedback path.

7. The interleaved amplifier for a sensing system of claim 6, wherein for the programmable gain, the first feedback path comprises a plurality of gain switching elements, each gain switching element comprising a switch in series with a resistor or a capacitor.

8. The interleaved amplifier for a sensing system of claim 7, wherein the first feedback path comprises a switched-capacitor network configured to define a direct current (DC) gain based on capacitor ratios.

9. The interleaved amplifier for a sensing system of claim 7, wherein the plurality of gain switching elements are rotated by a dynamic element matching (DEM) block when the first feedback path comprises a switch in series with a capacitor.

10. The interleaved amplifier for a sensing system of claim 9, wherein the DEM block generates a new pattern for each clock cycle and randomly selects one or more capacitors for charging, and wherein all the gain switching elements are used in any single gain setting to maintain a constant gain ratio among programmable gain settings.

11. The interleaved amplifier for a sensing system of claim 1, wherein an input interface to each amplifier of the N amplifiers comprises a single-ended input signal or a differential input signal.

12. The interleaved amplifier for a sensing system of claim 1, wherein an input signal to each amplifier of the N amplifiers comprises a voltage signal or a current signal.

13. The interleaved amplifier for a sensing system of claim 1, wherein an output of the first amplifier stage is sampled and added together at the second amplifier stage.

14. The interleaved amplifier for a sensing system of claim 1, wherein a filtering function comprising an anti-aliasing filter configured to attenuate high-frequency components prior to sampling is implemented in at least one of the first amplifier stage or the second amplifier stage.

15. The interleaved amplifier for a sensing system of claim 14, wherein programmable gain selection in the first amplifier stage comprises a plurality of parallel series paths, each series path comprising a feedback resistor and a gain switching element.

16. The interleaved amplifier for a sensing system of claim 14, wherein programmable gain selection in the first amplifier stage comprises a plurality of parallel series paths, each series path comprising a feedback capacitor and a gain switching element.

17. The interleaved amplifier for a sensing system of claim 14, wherein the anti-aliasing filter in the second amplifier stage comprises a switched capacitor low pass filter having a plurality of switching elements, a sampling capacitor, a holding capacitor, and a holding amplifier.

18. The interleaved amplifier for a sensing system of claim 1, wherein the sensor comprises a biopotential sensor for health monitoring applications.

19. The interleaved amplifier for a sensing system of claim 1, wherein the sensor comprises a photodiode.

20. The interleaved amplifier for a sensing system of claim 1, wherein the first amplifier stage operates as a transimpedance amplifier (TIA) to convert a current signal from to a voltage signal.

21. The interleaved amplifier for a sensing system of claim 1, wherein the interleaved operation of the first amplifier stage mitigates performance impact of reset operations by allowing at least one amplifier of the N amplifiers to continue processing the sensed signal while another amplifier of the N amplifiers undergoes reset.

22. The interleaved amplifier for a sensing system of claim 1, wherein each amplifier of the first amplifier stage comprises an integrator configured to be periodically reset or to operate as a leaky integrator, and wherein interleaving is configured to maintain continuous signal processing during such reset or leakage operation.

23. A method for operating an interleaved amplifier in a sensing system, the method comprising:
receiving, by an analog multiplexer (MUX), a sensed signal from a sensor;
selectively routing, by the analog MUX, the sensed signal to a selected amplifier of N amplifiers in a first amplifier stage, wherein N is an integer of two or greater, and wherein each of the N amplifiers is operable in an amplification mode and a sample and hold mode;
sampling, by a sample circuit block coupled to the first amplifier stage, an output of the selected amplifier;
holding, by a second amplifier stage comprising a hold amplifier block coupled to the sample circuit block, the sampled output; and
selecting, by the analog MUX, operation of only a single amplifier of the N amplifiers at a time such that the first amplifier stage operates as an interleaved amplifier providing increased bandwidth and reduced power consumption relative to a non-interleaved amplifier configuration.

24. The method of claim 23, wherein the first amplifier stage is configured to reduce gain at DC while maintaining gain within a target signal bandwidth.

25. The method of claim 23, wherein in the amplification mode, an input current or voltage from the analog MUX is amplified by the selected single amplifier, and wherein amplification by the selected single amplifier is achieved through integrating the input current or applying a voltage gain, the integration operation for the input current being implemented with a first feedback path of the selected single amplifier.

26. The method of claim 25, wherein in the sample and hold mode, an output of the selected single amplifier is sampled by the sample circuit block, and wherein the selected single amplifier transitions to a mode in which its output does not impact system performance after sampling.

27. The method of claim 26, wherein in a hold mode of the sample and hold mode, the second amplifier stage resets or switches operations of the selected single amplifier to a low power mode.

28. The method of claim 25, wherein each amplifier of the N amplifiers has a fixed gain or a programmable gain implemented in the first feedback path.

29. The method of claim 28, wherein for the programmable gain, the first feedback path comprises a plurality of gain switching elements, each gain switching element comprising a switch in series with a resistor or a capacitor.

30. The method of claim 29, wherein the first feedback path comprises a switched-capacitor network configured to define a DC gain based on capacitor ratios.

31. The method of claim 29, further comprising rotating, by a dynamic element matching (DEM) block, the plurality of gain switching elements when the first feedback path comprises a switch in series with a capacitor.

32. The method of claim 31, further comprising generating, by the DEM block, a new pattern for each clock cycle and randomly selecting one or more capacitors for charging, wherein all the gain switching elements are used in any single gain setting to maintain a constant gain ratio among programmable gain settings.

33. The method of claim 23, wherein an input interface to each amplifier of the N amplifiers comprises a single-ended input signal or a differential input signal.

34. The method of claim 23, wherein an input signal to each amplifier of the N amplifiers comprises a voltage signal or a current signal.

35. The method of claim 23, further comprising sampling an output of the first amplifier stage and adding the sampled outputs together at the second amplifier stage.

36. The method of claim 23, further comprising implementing a filtering function comprising anti-aliasing filtering (AAF) in at least one of the first amplifier stage or the second amplifier stage.

37. The method of claim 36, wherein programmable gain selection in the first amplifier stage comprises a plurality of parallel series paths, each series path comprising a feedback resistor and a gain switching element.

38. The method of claim 36, wherein programmable gain selection in the first amplifier stage comprises a plurality of parallel series paths, each series path comprising a feedback capacitor and a gain switching element.

39. The method of claim 36, wherein the AAF in the second amplifier stage comprises a switched capacitor low pass filter having a plurality of switching elements, a sampling capacitor, a holding capacitor, and a holding amplifier.

40. The method of claim 23, wherein the sensor comprises a biopotential sensor for health monitoring applications.

41. The method of claim 23, wherein the sensor comprises a photodiode.

42. The method of claim 23, wherein the first amplifier stage operates as a transimpedance amplifier (TIA) to convert a current signal to a voltage signal.

43. The method of claim 23, wherein the interleaved operation of the first amplifier stage mitigates performance impact of reset operations by allowing at least one amplifier of the N amplifiers to continue processing the sensed signal while another amplifier of the N amplifiers undergoes reset.

44. The method of claim 23, wherein each amplifier of the first amplifier stage comprises an integrator configured to be periodically reset or to operate as a leaky integrator, and wherein interleaving is configured to maintain continuous signal processing during such reset or leakage operation.
